(19) United States
(12) Patent Application Publication
(10) Pub. No.: US 2007/0098754 A1
(43) Pub. Date: May 3, 2007

(54) Method and article to restore function to GI or urinary tract of a patient

(75) Inventors: Yelena G. Tropsha, Plymouth, MN (US); Warren L. Starkebaum, Plymouth, MN (US)

Correspondence Address: IPLM GROUP, P.A., POST OFFICE BOX 18455, MINNEAPOLIS, MN, 55418, US

(73) Assignee: Medtronic, Inc.

(21) Appl. No.: 11/261,407

(22) Filed: Oct. 28, 2005

(51) Int. Cl. A61K 38/17; A61F 2/02

(52) U.S. Cl. 424/423; 514/2

(57) ABSTRACT

Method and article for restoring function to a region of a body selected from the group consisting of a gastrointestinal tract and a urinary tract of a patient. A biological agent is optionally associated with a carrier and inserted into the region of the body. The biological agent is stabilized in the region of the body. The biological agent is selected from a group consisting of nerve cells, interstitial cells of Cajal, stem cells and combinations thereof.

### RELATED APPLICATION

This disclosure is related to the following co-pending application entitled “Method and Article to Restore Function to a Flow Control Location in a Patient” by inventor Warren L. Starkebaum (Attorney Docket No. 151P21237US01), filed on even date herewith, which is not admitted as prior art with respect to the present disclosure by its mention in this section.

### FIELD OF THE INVENTION

The present invention is related to implantable medical devices and methods and, more particularly, to implantable medical devices and methods related to restoring function to the body of a patient.

### BACKGROUND OF THE INVENTION

Peristalsis is movement of the intestine which create waves of alternate circular contraction and relaxation by which the contents of the intestine are propelled forward. Such movement is a necessary for the proper functioning of the intestine in the movement of matter therein through the gastro-intestinal system of the body of a patient. Peristalsis is controlled by certain specialized cells called interstitial cells of Cajal which are present in networks throughout the gastro-intestinal tract.

In certain disorders, such cells may have been killed or disabled, for example by a viral infection. The result may be the disabling of normal peristaltic function of the gastro-intestinal tract. If such cells have been disabled in the stomach, the stomach may not contract normally, leading to a condition known as gastroparesis and the failure the gastro-intestinal track to function properly.

### BRIEF SUMMARY OF THE INVENTION

The present may allow the restoration of function of all or a portion of the gastro-intestinal tract and/or urinary tract of the body of a patient, especially when such gastro-intestinal tract has suffered nerve or other disorder preventing normal function.

In general, such function may be restored by introducing a biological agent into the region of the body affecting such dysfunction in order to reconstitute the nervous system disease of that region, especially the enteric nervous system affecting normal peristalsis. Such biological agent may be introduced in the wall of the gastro-intestinal tract or urinary tract.

The biological agent may be interstitial cells of Cajal, or stem cells, or combinations thereof.

The biological agent may be incorporated in a polymer matrix, which may be biodegradable or non-biodegradable, which can provide a slow release of such biological agent into the surrounding tissue.

In an embodiment, the present invention provides a method of restoring function to a region of a body selected from the group consisting of a gastrointestinal tract and a urinary tract of a patient. A biological agent is inserted into the region of the body. The biological agent is stabilized in the region of the body. The biological agent is selected from a group consisting of nerve cells, interstitial cells of Cajal, stem cells and combinations thereof.

In another embodiment, the biological agent is associated with a carrier.

In another embodiment, the tissue is muscle tissue.

In an embodiment, the present invention provides an article intended to restore a function to a region of a body of a patient selected from the group consisting of a gastrointestinal tract and a urinary tract. A carrier is adapted to be inserted into the region of the body. A biological agent, associated with the carrier, is selected from a group consisting of nerve cells, interstitial cells of Cajal, stem cells and combinations thereof.

In another embodiment, the biological agent is injected into tissue of the region of the body.

In another embodiment, the carrier is a non-biodegradable polymer matrix.

In another embodiment, the carrier is a plurality of non-biodegradable beads.

In another embodiment, the carrier is a plurality of non-biodegradable stars.

In another embodiment, the carrier is a hydrogel.

In another embodiment, the region is a location selected from the group consisting of a wall of a stomach and a wall of a small intestine.

### BRIEF DESCRIPTION OF THE DRAWINGS

FIG. 1 is a flow chart illustrating a method according to an embodiment of the invention;

FIG. 2 is a flow chart illustrating a method according to an alternative embodiment of the present invention;

FIG. 3 illustrates a biological agent associated with a polymer matrix carrier; and

FIG. 4 illustrates a biological agent associated with a dehydrated hydrogel.

### DETAILED DESCRIPTION OF THE INVENTION

The function of the gastro-intestinal tract and/or urinary tract, or a portion thereof, whose function has been disrupted by neuropathy or other nerve damage or other damage, may be restored by injecting a biological agent into tissue at or near the affected area. Such biological agent may be stabilized in the region of interest where such biological agent may grow in-situ to replace or augment natural smooth nerve that has been damaged and/or rendered non-responsive.

The function may be partially or fully restored with the administration of a biological agent. Biological agents which may be utilized for this purpose include, but are not limited to, nerve cells, interstitial cells of Cajal and stem cells, or combinations thereof.

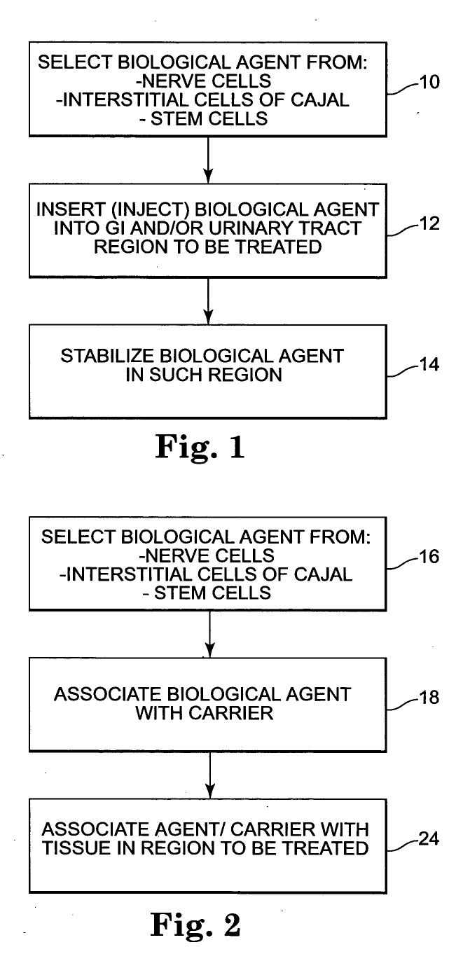

As indicated in the flow chart of FIG. 1, the biological agent may be selected (10) from several among the several classes of agents, including, but not limited to, those suggested above.

The biological agent is then inserted (12) into a region near the affected location in the gastro-intestinal tract and/or urinary tract. The location may, in fact, be at the location or in the location affected. However, beneficial results may be obtained even if the insertion location is not exactly at the affected location but rather in a location nearby. By “nearby” it is meant a location in the vicinity of the location at which the biological agent provides therapeutic effect to the affected region. The exact nearness depends, at least in part, on the particular portion of the gastro-intestinal tract and/or urinary tract to be restored.

Insertion of the biological agent can be accomplished by any number of conventional insertion techniques such as, for example, endoscope.

Once the biological agent has been inserted, the biological agent is stabilized (14) at the desired location. Stabilization of the biological agent can be accomplished by any number of conventional techniques. Examples of stabilization techniques that can be used are described in U.S. Patent Application Publication No. 2005/0096497, Gerber et al, Implantable Devices and Methods For Treating Fecal Incontinence, and U.S. Patent Application Publication No. 2005/0096751, Gerber et al, Implantable Devices and Methods For Treating Urinary Incontinence. Other techniques that can be used are described in U.S. Pat. No. 6,754,536, Swoyer et al, Implantable Medical Device Affixed Internally Within the Gastrointestinal Tract; U.S. Pat. No. 6,952,613, Swoyer et al, Implantable Gastrointestinal Lead With Active Fixation; U.S. Patent Application Publication No. 2002/0103521, Implantable Gastrointestinal Lead With Active Fixation; and U.S. Patent Application Publication No. 2002/0103522, Swoyer et al, Implantable Bifurcated Gastrointestinal Lead With Active Fixation. The contents of all of these documents are hereby incorporated by reference in their entirety.

FIG. 2 is a flow chart illustrating an alternative embodiment of a method of utilizing a biological agent to restore function to all or a portion of the gastro-intestinal tract and/or urinary tract having less than a fully responsive nervous system. Again biological agents which may be utilized for this purpose include, but are not limited to, nerve cells, interstitial cells of Cajal and stem cells, or combinations thereof. One or more of these biological agents, or another biological agent, or a combination is selected (16) for use in the restoration process.

The biological agent is then associated (18) with a carrier. A carrier is a structure which acts to carry or harbor the biological agent and facilitate the delivery and stabilization of the biological agent to a location at or near the affected location in the gastro-intestinal tract and/or urinary tract.

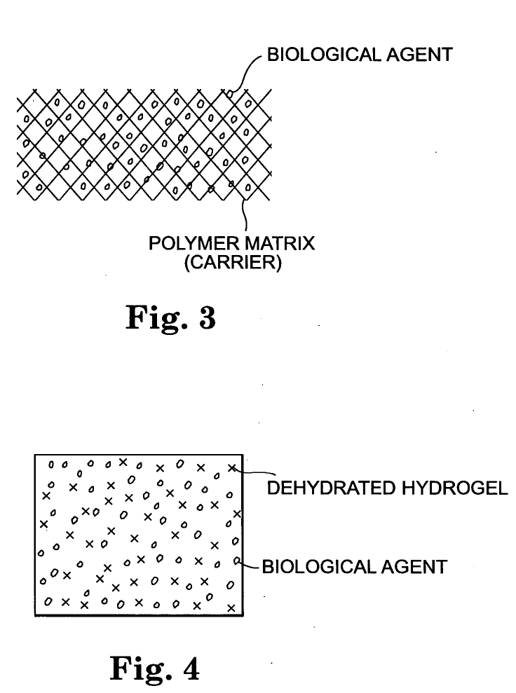

Carrier 20 illustrated in FIG. 3 is an example of a carrier that could be used for this purpose. Carrier 20 consists of a polymer matrix, preferably a biodegradable polymer matrix.

Biological agent 22 is associated with carrier 20 by, for example, saturating or impregnating carrier 20 with biological agent 22. Carrier 20 provides a physical carrier for biological agent 22 and allows biological agent 22 to be inserted into the body at the desired location at or near the flow control organ to be restored.

In a preferred embodiment illustrated in FIG. 4, carrier 20a consists of hydrogel. Carrier 20a may be filled or otherwise combined with biological agent 22. Carrier 20a and biological agent 22 combination may then be inserted into the body at the desired location at or near the flow control organ to be restored. The hydrogel may be dehydrated prior to insertion. When carrier 20 consisting of hydrogel is inserted into the body, the hydrogel will hydrate in situ.

Once biological agent 22 is associated (18 in FIG. 2), the combination is then associated (24) with tissue in the region of the gastro-intestinal tract and/or urinary tract to be restored. The combination may be physically placed at the desired site by any known and commonly available insertion technique as, for example, by use of an endoscope. Of course, it will be recognized and understood that will an endoscope may be the tool of choice for certain gastro-intestinal locations such as stomach, that other well known medical tools may be utilized for these locations as well as other locations outside of the upper gastrointestinal tract.

Biological agent 22 may be placed into tissue at the desired region by conventional means. In a preferred embodiment, biological agent 22 is placed in muscle tissue in such region.

The contents of co-pending application entitled “Method and Article to Restore Function to a Flow Control Location in a Patient” by inventor Warren L. Starkebaum (Attorney Docket No. 151P21237US01), filed on even date herewith, is hereby incorporated by reference in its entirety.

Thus, embodiments of the method and article to restore function to gastrointestinal or urinary tract of a patient are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A method of restoring function to a region of a body selected from the group consisting of a gastrointestinal tract and a urinary tract of a patient, comprising the steps of:
inserting a biological agent into said region of said body; and
stabilizing said biological agent in said region of said body;
wherein said biological agent is selected from a group consisting of nerve cells, interstitial cells of Cajal, stem cells and combinations thereof.

2. A method as in claim 1 wherein said inserting step comprises the step of injecting said biological agent into tissue of said region of said body.

3. A method as in claim 1 wherein said biological agent is associated with a carrier.

4. A method as in claim 3 wherein said carrier comprises a polymer matrix.

5. A method as in claim 4 wherein said polymer matrix comprises a non-biodegradable polymer matrix.

6. A method as in claim 4 wherein said polymer matrix comprises a biodegradable polymer matrix.

7. A method as in claim 3 wherein said carrier comprises a plurality of non-biodegradable beads.

8. A method as in claim 3 wherein said carrier comprises a plurality of non-biodegradable stars.

9. A method as in claim 3 wherein said carrier comprises a hydro-gel.

10. A method as in claim 1 wherein said region comprises a location selected from a group consisting of a wall of a stomach, a wall of a small intestine, an esophagus, a lower esophageal sphincter, an upper esophageal sphincter, a pylorus, a large intestine and an anal sphincter.

11. A method as in claim 1 wherein said stabilizing step comprises placing said biological agent into tissue near said region.

12. A method as in claim 11 wherein said tissue comprises muscle tissue.

13. An article intended to restore a function to a region of a body of a patient selected from the group consisting of a gastrointestinal tract and a urinary tract, comprising:
a carrier adapted to be inserted into said region of said body; and
a biological agent, associated with said carrier, selected from a group consisting of nerve cells, interstitial cells of Cajal, stem cells and combinations thereof.

14. An article as in claim 13 wherein said carrier is injected into tissue at said region of said body.

15. An article as in claim 13 wherein said carrier comprises a non-biodegradable polymer matrix.

16. An article as in claim 13 wherein said carrier comprises a plurality of non-biodegradable beads.

17. An article as in claim 13 wherein said carrier comprises a plurality of non-biodegradable stars.

18. An article as in claim 13 wherein said carrier comprises a hydrogel.

19. An article as in claim 13 wherein said region comprises a location selected from a group consisting of a wall of a stomach and a wall of a small intestine.

20. An article intended to restore a function to a region of a body of a patient selected from the group consisting of a gastrointestinal tract and a urinary tract, comprising:
carrier means for insertion into said region of said body; and
biological agent means, associated with said carrier, for supplying a biological agent selected from a group consisting of nerve cells, interstitial cells of Cajal, stem cells and combinations thereof.
